(19) United States
(12) Patent Application Publication
(10) Pub. No.: US 2025/0101387 A1
(43) Pub. Date: Mar. 27, 2025

(54) METHODS FOR ANALYZING DNA USING ENHANCED PERFORMANCE CHROMATOGRAPHY SYSTEMS

(71) Applicant: Regeneron Pharmaceuticals, Inc., Tarrytown, NY (US)

(72) Inventors: Dingjiang Liu, Pleasantville, NY (US); Li Zhi, Tarrytown, NY (US); Shuai Li, Hartsdale, NY (US)

(21) Appl. No.: 18/895,758

(22) Filed: Sep. 25, 2024

Related U.S. Application Data
(60) Provisional application No. 63/540,259, filed on Sep. 25, 2023.

(51) Int. Cl. C12N 7/00; B01D 15/34; G01N 30/88

(52) U.S. Cl. CPC C12N 7/00; B01D 15/34; G01N 30/88; C12N 2750/14151; G01N 2030/8827

(57) ABSTRACT

The present inventions relate to improved and enhanced chromatographic systems for analyzing DNA, such as DNA released from AAV virions. The present inventions avoid past problems through the use of larger pore size media, different column flow rates and buffers comprising surfactants and increased concentrations of inorganic salts, and typically have column pressures of about 1450 to about 1550 psi.

This application claims the benefit of priority to U.S. Application Ser. No. 63/540,259, filed Sep. 25, 2023, which is hereby incorporated by reference.

### FIELD OF THE INVENTIONS

The present inventions relate to improved and enhanced chromatographic systems for analyzing DNA, such as DNA released from AAV virions.

### BACKGROUND OF THE INVENTIONS

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807:1-23 (2011). Recombinant AAVs have demonstrated promise for human gene therapy based on their safety profile and potential to achieve long-term efficacy in animal models and in patients. Wang et al., Nature, 18:358-78 (2019). Recombinant AAVs include at least one gene of interest (GOI) along with AAV inverted terminal repeats (ITR) flanking the gene of interest.

SEC-UPLC has been used for analyzing free DNA in AAV viral preparations. See Xu et al., Int'l J. Pharmaceutics 615:121464 (2022). Other approaches, such as free DNA assays using fluorescent dye, have also been employed. See Bee et al., Int'l J. Pharmaceutics 110:3183-87 (2021).

A previous approach used an SEC column 5 μm, 4.6×300 nm with a 500 angstrom (Å) pore size. A preparation comprising 3.7×10¹² vector genomes (vg) in an injection volume was loaded onto the column using a running buffer based on 2× Dulbecco's Phosphate Buffered Saline (DPBS) at flow rate of 0.5 ml/minute (min.). The buffer had a salt concentration of 140 mM (millimolar).

This previous SEC-UPLC methodologies exhibited inconsistent performance and was limited by issues such as column clogging, pressure accumulations up to about 1700 psi, and limited resolution between peaks. Thus, this older approach cannot provide reliable characterization and quantitation of free DNA in AAV preparations. The present inventions address and overcome these limitations.

### SUMMARY OF THE INVENTIONS

The inventions provide methods of analysing free DNA in a preparation comprising AAV viral particles, wherein the method comprises the steps of: (a) loading the preparation comprising AAV viral particles in a size exclusion chromatography column having a pore size greater than 500 angstroms, wherein prior to loading the preparation is mixed with a running buffer; (b) running the preparation on the size exclusion chromatography column at a flow rate of less than 0.5 ml/minute, wherein the pressure in the size exclusion chromatography column does not accumulate and result in column clogging; and (c) detecting the elution of free AAV DNA and AAV viral particles from the size exclusion chromatography column.

The inventions provide methods, wherein the preparation of rAAV viral particles comprising a buffer, a cryoprotectant, a salt, a non-ionic surfactant at a pH of about 6.0 to about 8.0. The cryoprotectant can be sucrose at a concentration of about 0.5 to about 5.0% (w/v). The concentration of sucrose can be about 1.0 to about 3.5% (w/v), about 1.0 to about 3.0% (w/v), about 1.0 to about 2.5% (w/v), about 1.0 to about 2.0% (w/v), about 1.0 to about 1.5% (w/v), about 1.0% (w/v), about 1.5% (w/V), about 2.0% (w/v), about 2.5% (w/v), about 3.0% (w/v), as well as additional subranges made based upon the above values.

The size exclusion chromatography column media can have a nominal pore size of above 500 angstroms (Å), such as about 550 Å, about 600 Å, about 650 Å, about 700 Å, about 800 Å, about 850 Å, about 900 Å, 950 Å, about 1000 Å, about 1050 Å, about 1100 Å, about 1150 Å, about 1200 Å, about 1250 Å, about 1300 Å, about 1350 Å, about 1400 Å, about 1450 Å, or about 1500 Å. Ranges of pore size include, but are not limited to, 550 Å to 1500 Å, 700 Å to 1500 Å, 550 Å to 1100 Å, 700 Å to 1100 Å or 900 Å to 1100 Å, as well as additional subranges made based upon the above values.

Flow rates should be less than 0.5 ml/min to attenuate pressure accumulation. Preferably, average flow rates are about 0.49 ml/min, 0.48 ml/min, 0.47 ml/min, 0.46 ml/min, 0.45 ml/min, 0.44 ml/min, 0.43 ml/min, 0.42 ml/min, 0.41 ml/min, 0.40 ml/min, 0.39 ml/min, 0.38 ml/min, 0.37 ml/min, 0.36 ml/min, 0.35 ml/min, 0.34 ml/min, 0.33 ml/min, 0.32 ml/min, 0.31 ml/min, 0.30 ml/min, 0.29 ml/min, 0.28 ml/min, 0.27 ml/min, 0.26 ml/min, 0.25 ml/min, 0.24 ml/min, 0.23 ml/min, 0.22 ml/min, 0.21 ml/min, 0.20 ml/min, 0.19 ml/min, 0.18 ml/min, 0.17 ml/min, 0.16 ml/min, 0.15 ml/min, 0.14 ml/min, 0.13 ml/min, 0.12 ml/min, 0.11 ml/min, or 0.10 ml/min. Ranges of any of the above flow rates can be employed. For example, flow rates can be 0.30 ml/min to 0.40 ml/min, 0.31 ml/min to 0.39 ml/min, 0.32 ml/min to 0.38 ml/min, 0.33 ml/min to 0.37 ml/min, 0.34 ml/min to 0.36 ml/min, 0.33 ml/min to 0.35 ml/min, 0.34 ml/min to 0.35 ml/min, 0.35 ml/min to 0.38 ml/min, 0.35 ml/min to 0.37 ml/min or 0.35 ml/min to 0.36 ml/min as well as additional subranges made based upon the above values.

The running buffer can comprise one or more non-ionic surfactant, such as poloxamers, preferably poloxamer 188. The running buffer also can have a salt content of over 140 mM, 150 mM, 160 mM, 170 mM, 180 mM, 190 mM, 200 mM, 210 mM, 220 mM, 230, mM, 240 mM, 250 mM, 260 mM, 270 mM, 280 mM, 290 mM, 300 mM, 310 mM, 320 mM, 330, mM, 340 mM, 350 mM, 360 mM, 370 mM, 380 mM, 390 mM or 400 mM. Salt content ranges can include, but are not limited to, 140 mM to 400 mM, 150 mM to 400 mM, 180 mM to 400 mM, 200 mM to 380 mM, 220 mM to 360 mM, 240 mM to 360 mM, 260 mM to 360 mM or 260 mM to 340 mM, as well as additional subranges made based upon the above values.

The running buffer can comprise an inorganic salt. Optionally, the running buffer can comprise two or more types of inorganic salts, such as sodium chloride and sodium phosphate.

### BRIEF DESCRIPTION OF THE FIGURES

FIG. 1 is a graph depicting the elution of recombinant AAV8 virions and the elution of free DNA detected using a photodiode array (PDA) measuring 260 nm absorbance. Stressed recombinant AAV virions (10 freeze-thaw cycles for AAV8) and control recombinant AAV (thawed from −80° C.) are depicted in this Figure.

FIG. 2 is a graph depicting the elution of recombinant AAV8 virions and the elution of free AAV DNA detected using a PDA measuring 260 nm absorbance and 280 nm absorbance.

FIG. 3 is a graph depicting the elution of recombinant AAV8 virions and the elution of free DNA detected using a PDA measuring 280 nm absorbance and an Acquity Fluorescence Detector at excitation wavelength 280 nm, emission wavelength 350 nm.

FIG. 4 is a graph depicting the elution of DNA from two samples of DNA from stressed recombinant AAV8 main peak detected using a PDA measuring 260 nm absorbance. The stressed samples that were subjected to a temperature treatment of 50° C. for 30 minutes, and 50° C. for 30 minutes followed by a DNase treatment at 37° C. for 30 minutes are shown in this Figure. The graph in FIG. 4 also depicts control recombinant AAV (thawed from −80° C.).

FIGS. 5A-5C show data from characterization of genome DNA leakage from AAVs. FIG. 5A is a bar graph depicting quantitation of genome DNA leakage from recombinant AAV8 samples with 7.5E+12 vg/mL genome titer subjected to freeze/thaw, agitation, and thermal stress conditions. The dotted line assists with comparison between test samples and control. FIG. 5B is a bar graph depicting quantitation of genome DNA leakage of recombinant AAV8 with 3.0E+13 vg/mL genome titer subjected to various freeze-thaw cycles. The dotted line assists with comparison between test samples and control. FIG. 5C is a bar graph depicting data from samples subjected to freeze/thaw stress conditions in the AAV formulation containing 1.5% w/v sucrose. X: times of cycle; D: day; W: week; F/T: freeze/thaw. For each data point, n=6 (triplicated samples were prepared for each condition, and duplicated runs were performed for each sample).

FIG. 6 is graph showing genome DNA ejection and thermal melting profile of AAV8 measured by differential scanning fluorimetry (DSF) using SYBR gold dye. The dashed line indicates Tₒₙₛₑₜ for genome DNA ejection; the solid line indicates Tₘ.

FIGS. 7A-7B depicts data on evaluation of SEC free DNA method performance. FIG. 7A is a graph that shows the linearity between the AAV sample injection volume and the calculated free DNA amount. FIG. 7B is a graph showing reproducibility of the method by testing the same 10 freeze/thaw cycle stressed AAV sample over a two-month period.

FIG. 8 is a graph that shows there is a linear relationship between 2 kilobase (2 kb) double stranded (ds) DNA mass (in nanograms (ng)) and liquid chromatography (LC) Peak area.

FIG. 9 is a bar graph depicting free DNA concentrations (μg/ml) calculated for samples with varying injection volumes (μl).

FIG. 10 is a bar graph depicting quantitation of genome DNA leakage by SEC free DNA method in AAV5 samples before and after 4 cycles of freeze/thaw, and in formulations with and without 1.5% w/v sucrose. X: times of cycle; F/T: Freeze/Thaw.

FIG. 11 is a graph showing linearity between the reported Percent Full (% Full) capsids and the 260 nm/230 nm absorbance ratio. Stars indicate the two testing samples with theoretical % Full values reported using an orthogonal method.

FIGS. 12A-12B: FIG. 12A is a graph depicting SEC chromatogram of an AAV5 sample containing dimers (arrow) detected with 280 nm absorbance. FIG. 12B is a graph depicting characterization of AAV5 dimers by the SEC free DNA method using fluorescence detection with excitation at 280 nm and emission at 350 nm for a control AAV5 sample and an AAV5 sample after DNase treatment.

FIG. 13 is a graph depicting linearity between the AAV sample injection volume and the dimer peak absorbance area at 230 nm.

### DETAILED DESCRIPTION OF THE INVENTIONS

Unless defined otherwise, all technical and scientific terms used herein have the same meaning as commonly understood by one of ordinary skill in the art to which this invention belongs.

### Definitions

The term “about” in the context of numerical values and ranges refers to values or ranges that approximate or are close to the recited values or ranges such that the invention can perform as intended, such as having a desired rate, amount, density, degree, increase, decrease, percentage, value or presence of a form, variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Recombinant AAV vector” refers to an AAV virus that includes at least one gene of interest (GOI) along with AAV inverted terminal repeats (ITRs) flanking the GOI.

“Free DNA” refers to DNA species that are not bound to nor within viral capsids, and can be separated from viral capsids using SEC-UPLC.

A “gene of interest” (GOI) encodes a “protein of interest.” “Protein of interest” or “polypeptide of interest” (POI) can have any amino acid sequence, and includes any protein, polypeptide, or peptide that is desired to be expressed. Included are, but not limited to, viral proteins, bacterial proteins, fungal proteins, plant proteins and animal (including human) proteins. Protein types can include, but are not limited to, antibodies (including derivatives and fragment, receptors, Fc-containing proteins, trap proteins (including mini-trap proteins), fusion proteins, antagonists, inhibitors, enzymes (such as those used in enzyme replacement therapy), factors, repressors, activators, ligands, reporter proteins, selection proteins, protein hormones, protein toxins, structural proteins, storage proteins, transport proteins, neurotransmitters and contractile proteins. Derivatives, components, domains, chains and fragments of the above also are included. The sequences can be natural, semi-synthetic or synthetic.

An “inorganic salt” is a salt that lacks carbon-hydrogen bonds, and can be halide salt of an alkaline metal or alkaline earth metal. Inorganic salts include beryllium, lithium, sodium, and potassium salts of acetate; sodium and potassium bicarbonates; lithium, sodium, potassium, and cesium carbonates; lithium, sodium, potassium, cesium, and magnesium chlorides; sodium and potassium fluorides; sodium, potassium, and calcium nitrates; sodium and potassium phosphates; and calcium and magnesium sulfates. Inorganic salts can be combined in solution to form part of a buffer, such as a buffer comprising sodium chloride and sodium phosphate. Inorganic salts also can be combined with organic salts, which possess at least one carbon-hydrogen bond.

A “surfactant” is a substance which reduces the surface tension of a fluid in which it is dissolved and/or reduces the interfacial tension between two liquids, a liquid and a gas or a liquid and a solid. Surfactants can be ionic or non-ionic. Exemplary non-ionic surfactants that can be included in the formulations of the present invention include, e.g., alkyl poly(ethylene oxide), alkyl polyglucosides (e.g., octyl glucoside and decyl maltoside), fatty alcohols such as cetyl alcohol and oleyl alcohol, cocamide MEA, cocamide DEA, and cocamide TEA. Specific non-ionic surfactants that can be included in the formulations of the present invention include, for example, polysorbates such as polysorbate 20, polysorbate 28, polysorbate 40, polysorbate 60, polysorbate 65, polysorbate 80, polysorbate 81, and polysorbate 85; poloxamers such as poloxamer 188, poloxamer 407; polyethylene-polypropylene glycol; or polyethylene glycol (PEG). Polysorbate 20 is also known as TWEEN 20, sorbitan monolaurate and polyoxyethylenesorbitan monolaurate.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are intended to serve as a way of referring individually to each separate value falling within the range, unless otherwise indicated herein, and each separate value is incorporated into the specification as if it were individually recited herein. All recited numbers or values expressly set forth and denominate all ranges created by the recited numbers or values.

### DESCRIPTION

The present inventions provide a new SEC free DNA assay by optimizing the column property, mobile phase composition, and column regeneration procedure to improve the sensitivity and separation efficiency of the assay in order to achieve reliable quantitation of genome DNA leakage from AAV capsids. The optimized methods of the present inventions used a DNA standard to generate a calibration curve and achieved satisfactory assay performance in sensitivity, precision, and linearity for the quantitation of free DNA released from AAVs and proved applicable to multiple AAV serotypes. The inventions are amenable to all AAV serotypes including, for example, AAV1, AAV2, AAV2quad (Y-F), AAV2 7m8, AAV3, AAV4, AAV5, AAV6, AAV7, AAV8, AAV9, AAV10, AAV11, AAV12, AAV13, rh10, rh39, rh43, rh74, Avian AAV, Sea Lion AAV, Bearded Dragon AAV, as well as variants thereof. In addition, the present inventions demonstrate that the optimized SEC assay can also be applied to the characterization of multiple quality attributes of AAVs, including quantifying the percentage of full capsids and AAV dimers and can serve as an invaluable tool to support the development of AAV gene therapy products.

The present inventions provide improved performance over past approaches, which were hampered by column clogging, increased pressure accumulations up to about 1700 psi, and limited resolution between peaks. Column clogging and high pressures are believed to be a result of AAV virus particles adsorbing to the surface of chromatography medium, particularly for media having pores of 500 angstroms (Å) or smaller. The present inventions avoid these problems through the use of larger pore size columns, different column flow rates and buffers comprising surfactants and increased concentrations of inorganic salts, and typically have column pressures of about 1450 to about 1550 psi. For example, the column pressures can be about 1400 psi, about 1450 psi, about 1500 psi, about 1550 psi, about 1600 psi, about 1650 psi, or about 1700 psi.

According to the present inventions, the running buffers can comprise one on more inorganic salts at total salt concentrations of about 140 mM to about 400 mM, preferably about 180 mM to about 350 mM. For example, the running buffer also can have a salt content of over 140 mM, 150 mM, 160 mM, 170 mM, 180 mM, 190 mM, 200 mM, 210 mM, 220 mM, 230, mM, 240 mM, 250 mM, 260 mM, 270 mM, 280 mM, 290 mM, 300 mM, 310 mM, 320 mM, 330, mM, 340 mM, 350 mM, 360 mM, 370 mM, 380 mM, 390 mM or 400 mM. Preferred sub ranges include 200 mM to 350 mM, 225 to 350 mM, 250 nm to 350 mM, 300 mM to 350 mM, 305 mM to 345 mM, 310 mM to 340 mM, 315 mM to 335 mM, and 320 mM to 330 mM. The ranges scribed here expressly denominate and set forth all integers, decimals and fractional values defined and encompassed by the range. For example, 300 mM to 350 mM includes 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 mM, as well as fractional values there between.

One or more surfactants can be employed and used at total surfactant concentrations of about 0.001% to about 0.1% surfactant; or about 0.005% to about 0.05% surfactant. For example, the preparations of the present invention may comprise about 0.005%; about 0.006%; about 0.007%; about 0.008%; about 0.009%; about 0.010%; about 0.011%; about 0.012%; about 0.013%; about 0.014%; about 0.015%; about 0.016%; about 0.017%; about 0.018%; about 0.019%; about 0.020%; about 0.021%; about 0.22%; about 0.023%; about 0.024%; about 0.025%; about 0.026%; about 0.027%; about 0.028%; about 0.029%; about 0.030%, about 0.031%; about 0.32%; about 0.033%; about 0.034%; about 0.035%; about 0.036%; about 0.037%; about 0.038%; about 0.039%, 0.040%; about 0.041%; about 00.42%; about 0.043%; about 0.044%; about 0.045%; about 0.046%; about 0.047%; about 0.048%; about 0.049% or about 0.050% total surfactant.

Column loading concentrations of 10¹² to 10¹⁴ viral genomes/ml can be employed. Preferably, about 3.5×10¹² vg/ml to 9×10¹³ vg/ml. Preferred subrange include 4×10¹² vg/ml to 8×10¹³ vg/ml; 5×10¹² vg/ml to 7.5×10¹³ vg/ml; 6×10¹² vg/ml to 7×10¹³ vg/ml; 7×10¹² vg/ml to 7×10¹³ vg/ml; 7×10¹² vg/ml to 6×10¹³ vg/ml; 7.5×10¹² vg/ml to 5×10¹³ vg/ml; 7.5×10¹² vg/ml to 4×10¹³ vg/ml; 7.5×10¹² vg/ml to 3.5×10¹³ vg/ml; 7.5×10¹² vg/ml to 3×10¹³ vg/ml; 8×10¹² vg/ml to 3×10¹³ vg/ml; 9×10¹² vg/ml to 3×10¹³ vg/ml; or 1×10¹³ vg/ml to 3×10¹³ vg/m, for example.

Size exclusion chromatograph column media should have a pore size greater than 500 angstroms, often expressed by manufacturers as a nominal pore size. Preferably, the media has a pore size of about 550 Å, about 600 Å, about 650 Å, about 700 Å, about 800 Å, about 850 Å, about 900 Å, 950 Å, about 1000 Å, about 1050 Å, about 1100 Å, about 1150 Å, about 1200 Å, about 1250 Å, about 1300 Å, about 1350 Å, about 1400 Å, about 1450 Å, or about 1500 Å. The pore size should be above 500 angstroms (Å), such as about 550 Å, about 600 Å, about 650 Å, about 700 Å, about 800 Å, about 850 Å, about 900 Å, 950 Å, about 1000 Å, about 1050 Å, about 1100 Å, about 1150 Å, about 1200 Å, about 1250 Å, about 1300 Å, about 1350 Å, about 1400 Å, about 1450 Å, or about 1500 Å. Ranges of pore size include, but are not limited to, 550 Å to 1500 Å, 700 Å to 1500 Å, 550 Å to 1100 Å, 700 Å to 1100 Å or 900 Å to 1100 Å, as well as additional subranges made based upon the above values.

Flow rates should be less than 0.5 ml/min to attenuate pressure accumulation. Preferably, average flow rates are about 0.49 ml/min, 0.48 ml/min, 0.47 ml/min, 0.46 ml/min, 0.45 ml/min, 0.44 ml/min, 0.43 ml/min, 0.42 ml/min, 0.41 ml/min, 0.40 ml/min, 0.39 ml/min, 0.38 ml/min, 0.37 ml/min, 0.36 ml/min, 0.35 ml/min, 0.34 ml/min, 0.33 ml/min, 0.32 ml/min, 0.31 ml/min, 0.30 ml/min, 0.29 ml/min, 0.28 ml/min, 0.27 ml/min, 0.26 ml/min, 0.25 ml/min, 0.24 ml/min, 0.23 ml/min, 0.22 ml/min, 0.21 ml/min, 0.20 ml/min, 0.19 ml/min, 0.18 ml/min, 0.17 ml/min, 0.16 ml/min, 0.15 ml/min, 0.14 ml/min, 0.13 ml/min, 0.12 ml/min, 0.11 ml/min, or 0.10 ml/min. Ranges of any of the above flow rates can be employed. For example, flow rates can be 0.30 ml/min to 0.40 ml/min, 0.31 ml/min to 0.39 ml/min, 0.32 ml/min to 0.38 ml/min, 0.33 ml/min to 0.37 ml/min, 0.34 ml/min to 0.36 ml/min, 0.33 ml/min to 0.35 ml/min, 0.34 ml/min to 0.35 ml/min, 0.35 ml/min to 0.38 ml/min, 0.35 ml/min to 0.37 ml/min or 0.35 ml/min to 0.36 ml/min. Pressure accumulation refers to backpressure buildup inside the column. Column clogging means the column is clogged by particulates from sample or mobile phase and cannot function as usual or result in high column pressure.

### Buffers

The preparation of the rAAV viral particles comprises about 1 mM to about 20 mM, about 5 mM to about 15 mM, about 8 mM to about 12 mM, or about 10 mM±1 mM Tris buffer. The preparation can comprise about 1 mM to about 20 mM, about 5 mM to about 15 mM, about 8 mM to about 12 mM, or about 10 mM±1 mM sodium phosphate buffer. The preparation can comprise about 5 mM, about 6 mM, about 7 mM, about 8 mM, about 9 mM, about 10 mM, about 11 mM, about 12 mM, about 13 mM, about 14 mM, or about 15 mM Tris buffer. The preparation can comprise about 5 mM, about 6 mM, about 7 mM, about 8 mM, about 9 mM, about 10 mM, about 11 mM, about 12 mM, about 13 mM, about 14 mM, or about 15 mM sodium phosphate buffer. The preparation also can comprise about 10 mM±2 mM Tris buffer. The preparation can comprise about 10 mM±2 mM sodium phosphate buffer.

pH

The preparations of the rAAV viral particles of the present disclosure have a physiologically compatible pH. The preparations are provided that contain a buffering agent suitable to maintain the preparations at pH between about 6.0 and about 8.0. The pH of the preparations of the present disclosure can be about 6.0 to about 7.0, about 6.5 to about 7.5, about 6.6 to about 7.0, about 6.7 to about 7.0, about 6.8 to about 7.0, about 6.9 to about 7.0, about 7.0 to about 7.5, about 7.0 to about 7.1, or about 7.0 to about 7.3. The pH of the preparation can be about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. The pH of the preparations of the present disclosure can be about 7.0±0.1. The pH of the protein preparations of the present disclosure can be about 7.0±0.05. The pH of the protein preparations of the present disclosure can be about 6.9±0.1. The pH of the protein preparations of the present disclosure can be about 6.9±0.05.

### Cryoprotectants

The preparations of the rAAV viral particles of the present disclosure can include one or more cryoprotectants, such as one or more sugars.

Inclusion of one of more sugars (e.g., at between about 1% to about 10%) improves the stability of the preparations of the present disclosure. The preparations of the present disclosure can contain from about 1% to about 10% of one or more sugars. Any sugar such as mono-, di-, or polysaccharides, or water-soluble glucans, including for example fructose, glucose, mannose, mannitol, sorbose, xylose, maltose, lactose, sucrose, dextran, trehalose, pullulan, dextrin, cyclodextrin, soluble starch, hydroxyethyl starch, and carboxymethylcellulose can be used in the preparations. The sugar can be sucrose, trehalose, or a combination thereof.

The sugars can be used individually or in combination. The sugar, or a combination thereof, can be present in the preparations at a concentration of about 0.10% to about 1.0% (w/v), about 0.20% to about 1.0% (w/v), about 0.30% to about 1.0% (w/v), about 0.40% to about 1.0% (w/v), about 0.50% to about 1.0% (w/v), about 0.60% to about 1.0% (w/v), about 0.70% to about 1.0% (w/v), about 0.80% to about 1.0% (w/v), about 0.90% to about 1.0% (w/v), about 1.0% to about 10% (w/V), about 2% (w/v) to about 8% (w/v), about 2.5% to about 7.5% (w/v), about 3% (w/v) to about 7% (w/v), or about 4% to about 6% (w/v). The preparations of the present disclosure can comprise about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), about 3.0% (w/v), about 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4.0% (w/v), about 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), about 5.0% (w/v), about 5.1% (w/v), about 5.2% (w/v), about 5.3% (w/v), about 5.4% (w/v), about 5.5% (w/v), about 5.6% (w/v), about 5.7% (w/v), about 5.8% (w/v), about 5.9% (w/v), about 6.0% (w/v), about 6.1% (w/v), about 6.2% (w/v), about 6.3% (w/v), about 6.4% (w/v), about 6.5% (w/v), about 6.6% (w/v), about 6.7% (w/v), about 6.8% (w/v), about 6.9% (w/v), about 7.0% (w/v), about 7.1% (w/v), about 7.2% (w/v), about 7.3% (w/v), about 7.4% (w/v), about 7.5% (w/v), about 7.6% (w/v), about 7.7% (w/v), about 7.8% (w/v), about 7.9% (w/v), about 8.0% (w/v), about 8.1% (w/v), about 8.2% (w/v), about 8.3% (w/v), about 8.4% (w/v), about 8.5% (w/v), about 8.6% (w/v), about 8.7% (w/v), about 8.8% (w/v), about 8.9% (w/v), about 9.0% (w/v), about 9.1% (w/v), about 9.2% (w/v), about 9.3% (w/v), about 9.4% (w/v), about 9.5% (w/v), about 9.6% (w/v), about 9.7% (w/v), about 9.8% (w/v), about 19.9% (w/v), or about 10% (w/v) sugar.

The preparations of the present disclosure can include about 0.10% to about 1.0% (w/v), about 0.20% to about 1.0% (w/v), about 0.30% to about 1.0% (w/v), about 0.40% to about 1.0% (w/v), about 0.50% to about 1.0% (w/v), about 0.60% to about 1.0% (w/v), about 0.70% to about 1.0% (w/v), about 0.80% to about 1.0% (w/v), about 0.90% to about 1.0% (w/v), about 1.0% to about 10% (w/v), sucrose. The preparations can contain about 0.5% w/v±0.1%, about 1.0% w/v±0.1%, about 1.5% w/v±0.1%, or about 2.0% w/v±0.1% w/v sucrose.

### Salts

The preparations of the present disclosure can include one or more pharmaceutically acceptable salts.

The pharmaceutically acceptable salts can include, but are not limited to, metal salts such as sodium, potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) can include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; amino acid salts such as arginate, alaninate, asparginate and glutamate; and carbohydrate salts such as gluconate and galacturonate. Non-limiting examples of pharmaceutically acceptable salts include, without limitation, sodium salts, ammonium salts, potassium salts, calcium salts, and magnesium salts (e.g., sodium, ammonium, potassium, calcium, and magnesium chloride; sodium, ammonium, potassium, calcium and magnesium acetate; sodium, ammonium, potassium, calcium and magnesium citrate; sodium, ammonium, potassium, calcium and magnesium phosphate; sodium, ammonium, potassium, calcium and magnesium fluoride; sodium, ammonium, potassium, calcium and magnesium bromide; and sodium, ammonium, potassium, calcium and magnesium iodide). The pharmaceutically acceptable salt can be sodium chloride or arginine hydrochloride (L-arginine hydrochloride).

The preparations of the present disclosure can also include one or more pharmaceutically viscosity reducers such as sodium chloride, lysine, proline, and the like.

The preparations of the present disclosure can comprise about 10 mM to about 300 mM, about 50 mM to about 150 mM, about 50 mM to about 100 mM, about 50 mM to about 200 mM, about 50 mM to about 250 mM, about 50 mM to about 300 mM, about 100 mM to about 200 mM, about 100 mM to about 250 mM, about 100 mM to about 300 mM, 150 mM to about 200 mM, about 150 mM to about 250 mM, about 150 mM to about 300 mM, about 250 mM to about 300 mM, about 75 mM to about 100 mM, about 175 mM to about 200 mM, about 175 mM to about 225 mM, about 200 mM to about 225 mM, about 225 mM to about 275 mM, about 275 mM to about 300 mM, or about 175 mM to about 275 mM of a pharmaceutically acceptable salt. The preparations of the present disclosure can comprise about 0 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM of a pharmaceutically acceptable salt. The p preparations of the present disclosure can comprise about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, 106 mM, about 107 mM, about 108 mM, about 109 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, about 205 mM, about 210 mM, about 215 mM, about 220 mM, about 225 mM, about 230 mM, about 240 mM, about 245 mM, about 250 mM, about 255 mM, about 260 mM, about 265 mM, about 270 mM, about 275 mM, about 280 mM, about 285 mM, about 290 mM, about 295 mM, or about 300 mM, of a pharmaceutically acceptable salt.

The preparations of the present disclosure can comprise about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM of sodium chloride. The preparations of the present disclosure can comprise about 100 mM±5 mM, about 110 mM±5 mM, about 120 mM±5 mM, about 130 mM±5 mM, about 140 mM±5 mM, about 150 mM±5 mM, about 160 mM±5 mM, about 170 mM±5 mM, about 180 mM±5 mM, about 190 mM±5 mM, about 200 mM±5 mM, or about 210 mM±5 mM sodium chloride.

### Surfactants

The preparations of the present disclosure can include one or more surfactants.

The preparations of the present disclosure can contain a stabilizing concentration of a pharmaceutically acceptable non-ionic surfactant. Pharmaceutically acceptable non-ionic surfactants that can be used in the preparations discussed herein can include, without limitation, polysorbate 80 (Tween 80; PS80), polysorbate 81 (Tween 81; PS81), polysorbate 82 (Tween 82; PS82), polysorbate 20 (Tween 20; PS20), and various poloxamers (e.g., poloxamer 188), or mixtures thereof.

The preparations of the present disclosure can comprise from about 0.01% (w/v) to about 0.30% (w/v) non-ionic surfactant. The preparations can comprise about 0.01% to 0.30% (w/v), about 0.01% to 0.05% (w/v), about 0.05% to 0.10% (w/v), about 0.10% to 0.15% (w/v), about 0.15% to 0.20% (w/v), about 0.20% to 0.25% (w/v), or about 0.25% to 0.30% (w/v) non-ionic surfactant. The preparations of the present disclosure can comprise about 0.05% (w/v), about 0.06% (w/v), about 0.07% (w/v), about 0.08% (w/v), about 0.09% (w/v), about 0.10% (w/v), about 0.11% (w/v), about 0.12% (w/v), about 0.13% (w/v), about 0.14% (w/v), about 0.15% (w/v), about 0.16% (w/v), about 0.17% (w/v), about 0.18% (w/v), about 0.19% (w/v), about 0.20% (w/v), about 0.21% (w/v), about 0.22% (w/v), about 0.23% (w/v), about 0.24% (w/v), about 0.25% (w/v), about 0.26% (w/v), about 0.27% (w/v), about 0.28% (w/V), about 0.29% (w/v), or about 0.30% (w/v) non-ionic surfactant. The preparations of the present disclosure can comprise about 0.20% w/v±0.01% w/v non-ionic surfactant.

The preparations of the present disclosure can comprise about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), or about 0.01% (w/v) poloxamer 188. The preparations of the present disclosure can comprise about 0.005% w/v±0.001% w/v polysorbate 80 or about 0.005% w/v±0.001% w/v poloxamer 188.

The inventions are further described by the following Examples, which are supportive and illustrative of the inventions, but do not limit the inventions in any manner.

### EXAMPLES

### Materials and Reagents

Recombinant AAV8 and AAV5 viral vectors encapsulating a single-stranded DNA (ssDNA) transgene of approximately 4.0 kilo base (kb) were produced by triple transfection of HEK293 cells in suspension cell culture, purified using affinity chromatography followed by anion-exchange chromatography. Kolliphor® Poloxamer 188 (P188) BIO is from BASF (Geismar, LA). Tris base and Tris hydrochloride is from Avantor J.T. Baker® (Radnor Township, PA). All other excipients and reagents are of compendial grade. 2.0 kb double-stranded DNA (dsDNA) standard and Invitrogen™ DNase I is from ThermoFisher Scientific (Waltham, MA). The Crystal Zenith® (CZ) Cyclic Olefin Polymer (COP) vials, aluminum Flip-Off® seals, and serum NovaPure® stoppers are from West Pharmaceutical Services (Exton, PA).

### Sample Preparation

AAV8 and AAV5 vectors were formulated to a target titer of 7.5E+12 or 3E+13 vector genome (vg)/mL, respectively, in either formulation 1 (F1) containing 10 mM Tris, 180 mM sodium chloride, 0.005% w/v P188, pH 7.3 or formulation 2 (F2) containing 1.5% w/v sucrose in addition to the same excipients as those in F1. Upon completion of formulation, 0.4 mL of AAV8 or AAV5 vectors were filled into 2 mL CZ vials, sealed with 13 mm serum NovaPure stoppers along with 13 mm aluminum Flip-Off seals.

### Generation of Stressed AAV Samples

To generate freeze/thaw stressed AAV samples, the vials containing AAV8 and AAV5 were placed in a −80° C. freezer for at least 1.5 hours, then transferred to bench top at room temperature for at least 1 hour to complete each freeze/thaw cycle. To generate agitation stressed samples, the AAV8 and AAV5 vials were placed on an orbital shaker set at 300 rpm for 1 and 2 days. To generate thermal stressed samples, the AAV8 and AAV5 vials were incubated at 37° C. for 1 and 2 weeks, or at 50° C. for 30 minutes. A control AAV sample was kept at −80° C.

### DNase Treatment

To digest any non-encapsulated DNA that may present in the samples, AAV8 and AAV5 vectors were treated with DNase I by following the manufacturer's instructions. Briefly, 30 μL of DNase I Reaction Mix was added to 20 μL of an AAV sample in a polymerase chain reaction (PCR) tube. Following a 15-second gentle vortexing, the reaction mixture was incubated in a thermal cycler set at 37° C. for 30 minutes, then the temperature was lowered to 4° C. until analysis.

### Size-Exclusion Chromatography (SEC)

SEC was performed on a Waters™ ACQUITY UPLC system equipped with a photodiode array detector (PDA, absorbance wavelength at 230 nm, 260 nm, and 280 nm) and a fluorescence detector (FLD, excitation wavelength at 280 nm, emission wavelength at 350 nm). A SRT SEC column (1000 Å, 5 μm, 4.6×300 mm, Sepax Technologies, Newark, DE) was used for separating free DNA from AAVs. All sample vials were held in the auto-sampler set at 4° C. AAV samples were analyzed at a flow rate of 0.35 mL/minute using a mobile phase containing 20 mM sodium phosphate, 300 mM sodium chloride, 0.02% w/v P188, pH 7.3 with 40 minutes of total running time for each sample.

### Optimization of SEC Method

There were two objectives for optimizing the SEC method for quantitation of free DNA released from AAVs. The first objective was to prevent pressure accumulation or blockage in the column, which was identified to be the major issue with the previously reported SEC method, potentially due to the high surface adsorption tendency inherent to AAV samples. The second objective was to improve the separation efficiency between the free DNA peak and AAV peak.

In order to achieve these objectives, the current inventions optimized the mobile phase by including 300 mM sodium chloride and 0.02% w/v P188 to prevent accumulation of column pressure. High salt is generally included in SEC mobile phase, which can effectively prevent adsorption to the column by reducing charge-mediated nonspecific interactions. Furthermore, a mobile phase containing high salt can be more compatible with AAV sample analysis because high salt is useful for preventing AAV aggregation. Surfactants such as P188 function by reducing the surface tension at the interface to the stationary phase of the SEC column and hence can inhibit the adsorption of AAVs and free DNA due to competition with the surfactant for binding on the interface. Notably, 0.02% w/v P188 played a role for reducing adsorption on the column as a significant pressure accumulation was observed when it was omitted from the mobile phase or at a lower concentration such as 0.005% w/v. In addition, the column was optimized for running, regeneration, and storage conditions which have pronounced impacts on column performance such as causing a significant increase in back pressure or loss in separation resolution. For example, although the free DNA and the main AAV particle peaks eluted within 13 minutes of run time, the run time was extended to 40 minutes to ensure any residual level of precipitated proteins or other contaminants that can build up on the column and require extended elution time to be completed removed before initiation of subsequent injections. After completion of the SEC run, the column was flushed with 3 column volumes of water followed by 3 column volumes of 20% ethanol at a flow rate of 0.2 mL/minute to prevent microbial growth. By applying this optimized SEC protocol, excellent separation resolution of the two peaks with the free DNA peak eluting at about 7 minutes and the AAV particle peak at about 9.5 minutes were achieved. The identity of the free DNA peak has been verified using NGS as described in Xu et al (Xu et al. 2022. Genome DNA leakage of Adeno-Associated virus under freeze-thaw stress. Int J Pharm 615:121464).

As a result of the method optimization, a new and improved SEC free DNA method was developed which demonstrated good separation efficiency between the free DNA and AAV particle peaks. Enhanced accuracy and reproducibility of the assay were achieved without pressure accumulation. Moreover, the optimized mobile phase is relatively mild at neutral pH 7.3 and does not contain organic solvents, which matches the pH of the AAV formulations tested in experiments disclosed herein. Also, P188 at 0.02% w/v demonstrated good compatibility by several AAV serotypes. Thus, the optimized SEC mobile phase pose minimal or no risk for causing conformational change or denaturation of the AAV particles.

The inventions are further described by the following Examples, which do not limit the inventions in any manner and are applicable to all sections of the descriptions of the inventions and the aspects of the inventions. The order of performance of the below Examples can be altered or combined as determined by the person of skill in the art in view of the teachings and data contained herein.

### Example 1

This Example provides evidence that the inventions can detect and differentiate free DNA from AAV virions. Recombinant AAV8 at a concentration of 7.5 E+10¹² vg/ml in a storage buffer of 10 mM Tris, 180 mM NaCl, 0.005% Poloxamer 188 at a pH of 7.3 was subjected to 10 freeze-thaw cycles (10× Freeze/Thaw). Excess Freeze/Thaw stresses the viral capsids and results in capsid instability thereby releasing DNA. A control recombinant AAV 8 preparation was thawed without being subjected to repeat freeze-thaw cycles.

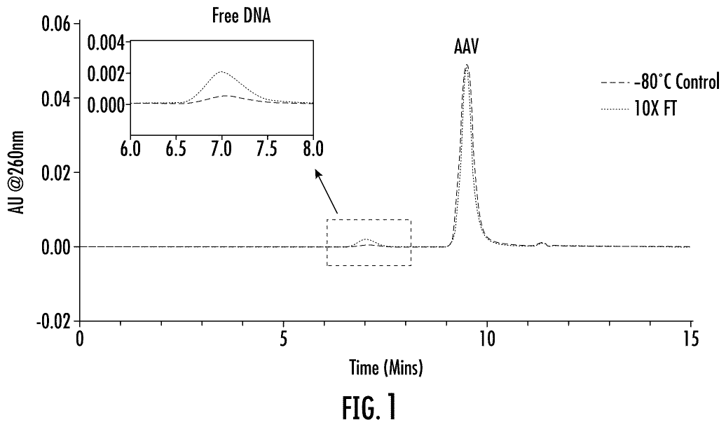

FIG. 1 is a representative of chromatography profiles collected with absorbance at 260 nm for an exemplary control AAV8 sample stored at −80° C. and an AAV sample after 10 cycles of freeze/thaw treatment. Baseline separation was achieved between the free DNA peak eluting at about 7 minutes and the main AAV particle peak eluting at about 9.5 minutes. Free DNA from the stressed sample and the control sample had sufficient retention time differences with the AAV main peak to be readily distinguished. Compared to the control AAV sample, significant increase in both the area and height of the free DNA peak was observed in the freeze/thaw stressed AAV sample. In addition, the signal-to-noise ratio of the free DNA peak detected in the stressed AAV sample was about 69.3, significantly higher than 10, suggesting the feasibility of applying this improved SEC method to the quantitation of released free DNA from AAV samples.

### Example 2

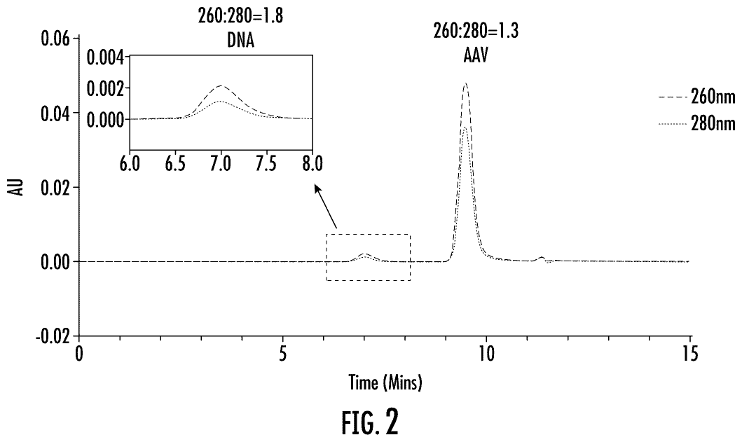

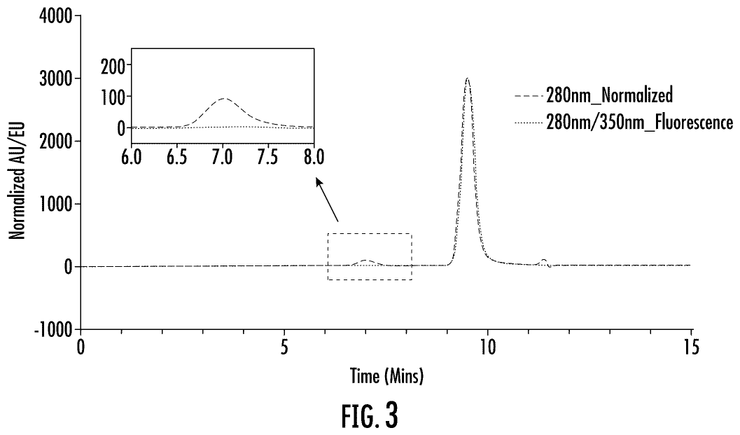

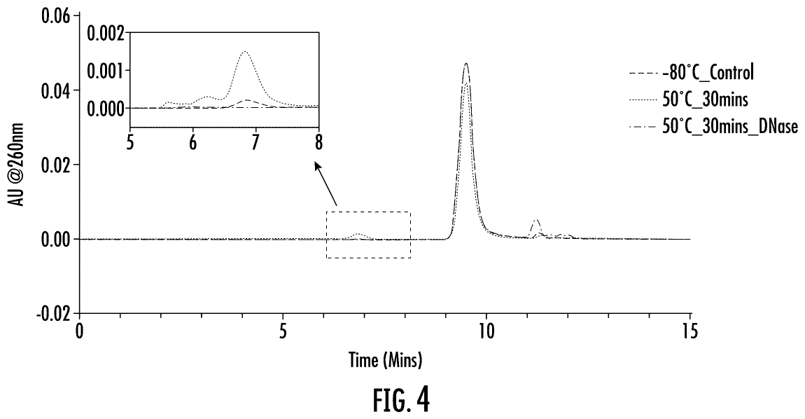

This Example provides the verification of free DNA peak. To verify the free DNA peak, three orthogonal methods were used. The AAV samples subjected to either 10 cycles of freeze/thaw (FIGS. 2 and 3) or 50° C. treatment (FIG. 4) were used in the assessment study. First, the purity of free DNA was accessed using the ratio of absorbance at 260 nm and 280 nm. A 260/280 ratio of about 1.8 is generally accepted as “pure” for DNA. If the ratio is appreciably lower, it indicates the presence of protein or other contaminants that absorb strongly at or near 280 nm. As shown in FIG. 2, the 260 to 280 (260/280) ratio for the main peak was 1.3, which is typical for AAV particles containing capsid proteins and genome DNA. In contrast, the 260/280 ratio for the free DNA peak was 1.8, indicating the peak is purely DNA. Second, since the intrinsic fluorescence of nucleic acids is extremely weak compared to the fluorescence of proteins, nucleic acids are often considered as non-fluorescent. The fluorescence signals with excitation at 280 nm and emission at 350 nm (FIG. 3) were therefore collected. Indeed, fluorescence signals were only detected at the main AAV particle peak but not at the free DNA peak, confirming the free DNA peak contains pure DNA without proteins. Lastly, the AAV8 sample was stressed at 50° C. for 30 minutes followed by treatment with DNase to remove any free DNA released from AAV capsids. As shown in FIG. 4, there was a pronounced increase of free DNA after thermal stress; after the DNase treatment, the level of free DNA decreased to approximately baseline, again confirming the free DNA peak contains pure DNA without proteins.

### Example 3

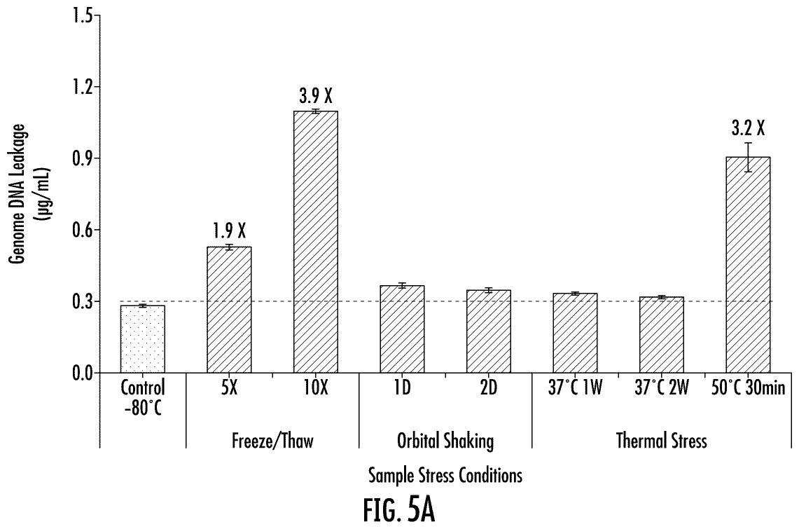

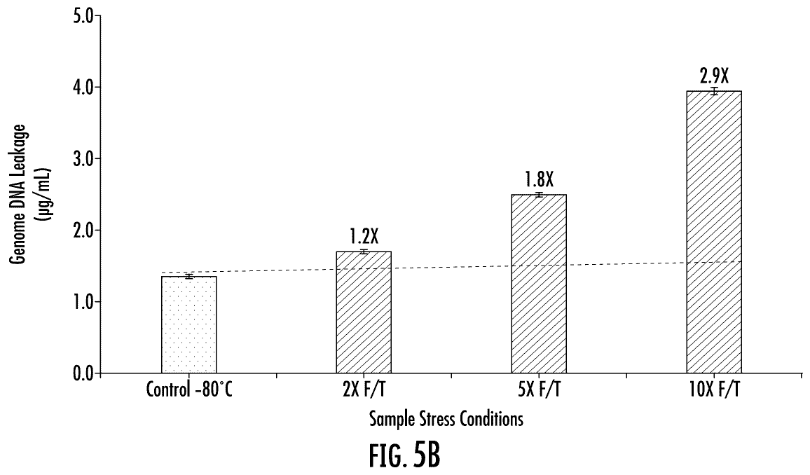

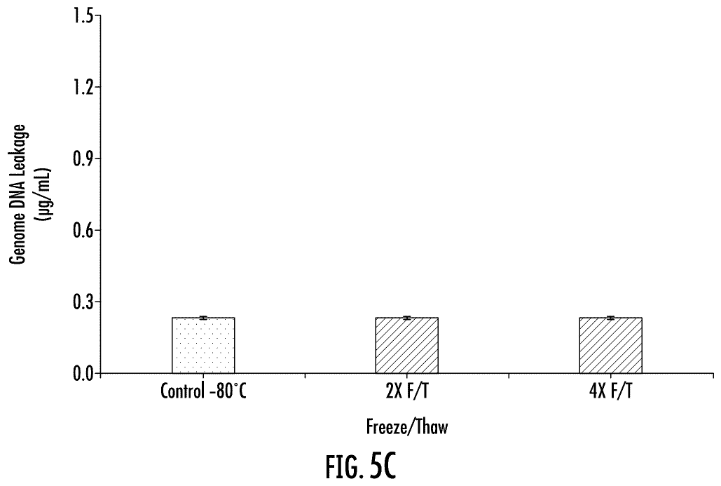

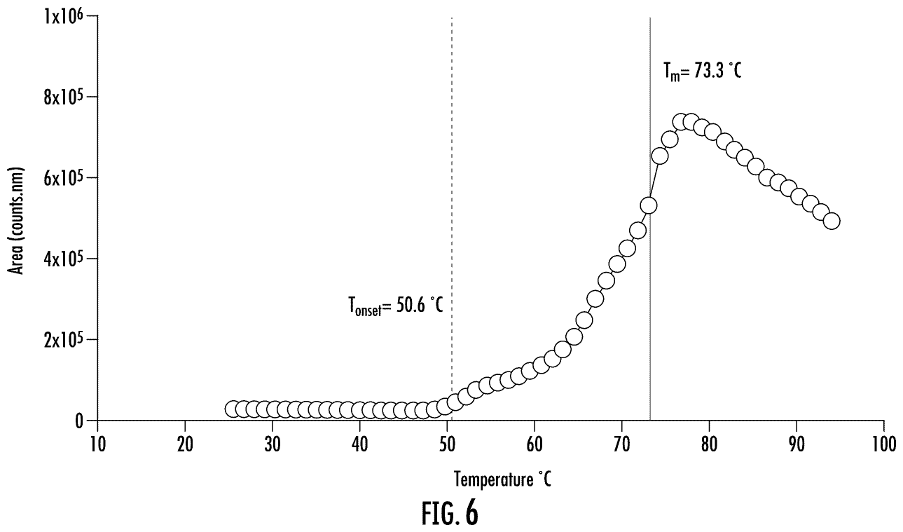

This Example concerns genome DNA leakage from AAVs under different stress conditions. Once the optimized SEC methods were established, this assay was applied to evaluate the genome DNA leakage for AAV8 under various stress conditions, including up to 10 cycles of freeze/thaw, up to 2 days of agitation, and up to 2 weeks of thermal treatment at 37° C. (FIG. 5A). In addition, an AAV8 sample treated for 30 minutes at 50° C. was included as a positive control since the onset temperature of genome ejection for AAV8 capsids was determined previously to be about 50° C. (FIG. 5A). The results from this experiment showed that AAV8 capsids were especially sensitive to freeze/thaw stress. Approximately 1.9-fold (from 0.28 μg/mL to 0.53 g/mL) and 3.9-fold (from 0.28 μg/mL to 1.10 μg/mL) increase in free DNA were observed after 5 and 10 freeze/thaw cycles, respectively. In contrast, neither agitation by orbital shaking nor thermal treatment at 37° C. induced substantial genome DNA release compared to the AAV8 sample stored at −80° C. The Freeze/thaw induced DNA leakage was confirmed with high genome titer samples, with 3.0E+13 vg/mL genome titer, subjected to various freeze-thaw cycles (FIG. 5B). Therefore, AAV stabilization during the freeze/thaw process becomes critical for mitigating the risks associated with genome DNA leakage. Data is shown below in Table 1 and depicted in a bar graph in FIG. 5A.

TABLE 1

| | | Average | | | Signal | |
|---|---|---|---|---|---|---|
| | | Free DNA | Calculated | Relative | to | |
| | | Peak | Free DNA | Standard | Noise | |
| | | Area | Concentration | Deviation | Ratio | |
| Stress Conditions | | (μV*Sec) | (μg/mL) | (RSD) | (S/N) | P-Value |
| Control | −80° C. | 1.59E+04 | 0.28 | 1.55% | 16.84 | |
| Freeze/Thaw | 5X | 2.99E+04 | 0.53 | 2.12% | 31.64 | <0.001 |
| | 10X | 6.21E+04 | 1.10 | 0.76% | 69.33 | <0.001 |
| Orbital | 1 day | 2.07E+04 | 0.37 | 3.00% | 20.15 | <0.001 |
| Shaking | 2 days | 1.96E+04 | 0.35 | 2.84% | 19.92 | <0.001 |
| Thermal | 37° C. 1 W | 1.88E+04 | 0.33 | 1.30% | 7.78 | <0.001 |
| Stress | 37° C. 2 W | 1.80E+04 | 0.32 | 1.31% | 7.49 | <0.001 |
| | 50° C. | 5.12E+04 | 0.91 | 6.69% | 58.36 | <0.001 |
| | 30 min | | | | | |

### Example 4

This Example tested recombinant AAV8 virions at 3×10¹³ vg/ml to 2, 5 or 10 cycles of Freeze/Thaw compared to a control. Freeze/Thaw, which stresses the viral capsids, results in capsid instability, thereby releasing more DNA as Freeze/Thaw cycles increase.

Data is shown below in Table 2 and depicted in a bar graph in FIG. 5B.

TABLE 2

| | Average | Calculated | Relative | Signal to | |
|---|---|---|---|---|---|
| | Free DNA | Free DNA | Standard | Noise | |
| Stress | Peak Area | Concentration | Deviation | Ratio | P- |
| Conditions | (μV*Sec) | (μg/mL) | (RSD) | (S/N) | Value |
| Control - | 7.76E+04 | 1.37 | 1.96% | 91.89 | |
| 80° C. | | | | | |
| 2X F/T | 9.65E+04 | 1.71 | 1.13% | 110.04 | <0.001 |
| 5X F/T | 1.42E+05 | 2.51 | 0.74% | 164.27 | <0.001 |
| 10X F/T | 2.23E+05 | 3.95 | 1.02% | 257.15 | <0.001 |

This example demonstrated that Free AAV DNA increases along with increase cycles of Freeze/Thaw.

### Example 5

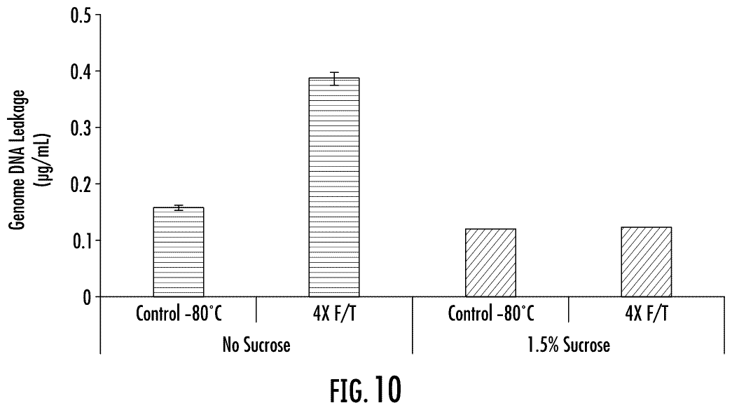

This Example shows the mitigation of genome DNA leakage with cryoprotectants. To design a robust and stable AAV formulation, an important decision is to select excipients based on their properties and established mechanisms for protein stabilization. Cryoprotectants, in particular sucrose, are selected as excipients to stabilize proteins, especially for those sensitive to freeze/thaw stress. Cryoprotectants function by various mechanisms including preferential exclusion from protein surface, forming glassy matrix around protein molecules, or forming hydrogen bonds with proteins. Surfactants such as P188 are also commonly selected for AAV formulations. As described herein, surfactants reduce the surface tension at the interface created by mechanical disturbance or freeze/thaw process, and hence inhibit the adsorption of proteins at the interface or the surface-induced protein denaturation due to competition with the surfactant for binding on the interface. Since the AAV formulations evaluated in this study contain P188 at a level similar to what is commonly used in clinical and approved AAV products (0.005% w/v P188 in F1), the AAV8 samples were formulated with a buffer containing 1.5% w/v sucrose while maintaining P188 at the same level (F2). The concentration of sucrose was selected to provide sufficient stabilization while maintaining isotonicity of the formulation in order to accommodate the desired route of administration such as intravenous delivery. The AAV8 samples in the 1.5% w/v sucrose-containing formulation were subjected to up to 4 freeze/thaw cycles followed by analysis of the free DNA using the SEC method (FIG. 5C). Compared to the AAV8 sample in formulations without sucrose, which showed a 1.9-fold increase after 5 freeze/thaw cycles (FIG. 5A), the addition of 1.5% w/v sucrose in the AAV8 formulation effectively mitigated genome DNA release due to freeze/thaw stress, thus no apparent increase in free DNA was observed after 4 freeze/thaw cycles. Similar results have been observed with AAV5 (FIG. 10), confirming 1.5% sucrose is sufficient for mitigating the potential risks associated with genome DNA leakage under freeze/thaw stress, and can be included in AAV formulations intended for long-term frozen storage.

### Example 6

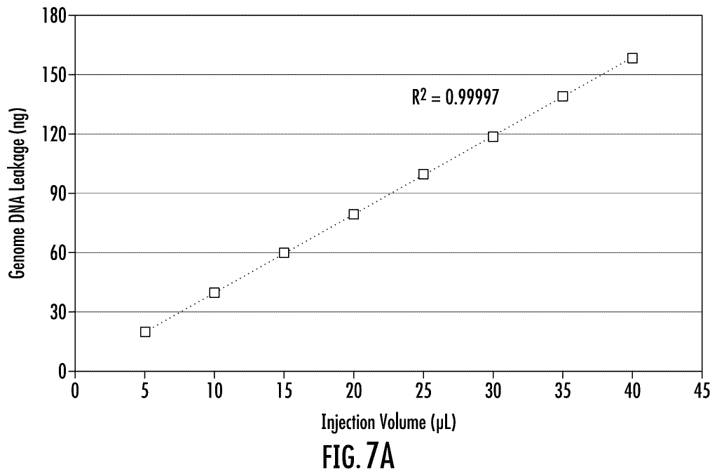

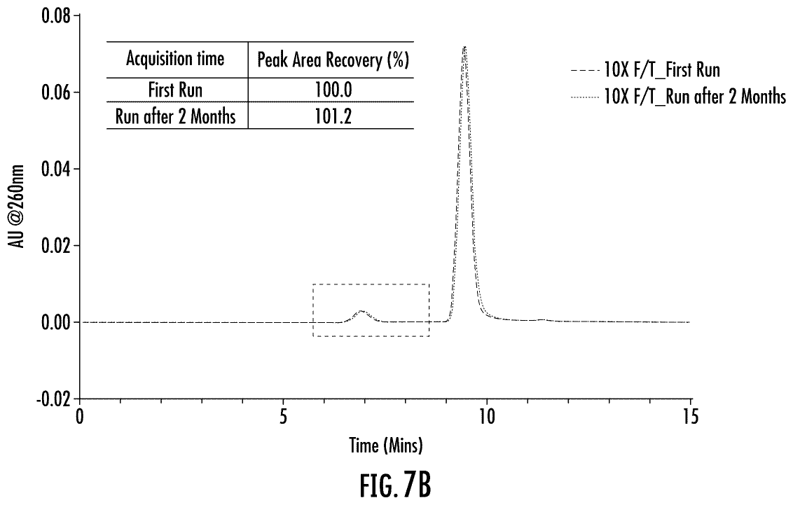

This Example shows the evaluation of SEC method performance. In order to access whether the optimized SEC method is sufficient for quantitation of free DNA, the method performance was characterized with a focus on linearity, sensitivity, and precision using an AAV8 sample at 3E+13 vg/mL stressed with 10 freeze/thaw cycles. A series of six injections with increasing volume from 5 μl to 40 μL were performed. Then, the amount of released genome DNA was calculated following the formula described above and plotted against the injection volume. As shown in FIG. 7A, a strong linear correlation was observed with a R2 value of 0.99997 after linear regression analysis. Data was also shown in Table 3. Furthermore, the signal-to-noise ratio was larger than 10 for the free DNA peak in samples with the minimal injection volume of 5 μL, suggesting satisfactory sensitivity of the optimized SEC method for quantitation of the released genome DNA.

TABLE 3

| Injection volume | Average Free DNA | Signal to Noise | Free DNA |
|---|---|---|---|
| (μL) | Peak Area (μV*Sec) | Ratio (S/N) | (ng) |
| 5 | 3.73E+04 | 43.8 | 19.8 |
| 10 | 7.45E+04 | 85.5 | 39.5 |
| 15 | 1.13E+05 | 145.7 | 59.9 |
| 20 | 1.49E+05 | 162.6 | 79.1 |
| 25 | 1.88E+05 | 196.4 | 99.4 |
| 30 | 2.24E+05 | 250.6 | 118.7 |
| 35 | 2.62E+05 | 310.5 | 139.0 |
| 40 | 2.99E+05 | 328.0 | 158.4 |

To evaluate the precision of the optimized SEC method, six replicates of each of the AAV8 samples at 7.5E+12 vg/mL stressed under various conditions were measured (FIG. 5A). The coefficient of variance (CV) was determined for each stressed AAV8 sample and found to be within 7%, demonstrating excellent repeatability of the method. Next, the intermediate precision of the method was accessed by measuring the same freeze/thaw stressed AAV8 sample over a two-month period. The results, as shown in FIG. 7B, indicated a highly consistent profile of the free DNA peak with a negligible 1% difference. Overall, the results demonstrate that the optimized SEC method is capable for providing reliable results for the quantitation of free DNA, or in another words, the genome DNA leakage from AAV capsids. These stress studies also were performed with AAV5 at approximately the same target titers (FIG. 10). Similar levels of genome DNA leakage were found as observed with AAV8. This optimized SEC method was also used to characterize the genome DNA leakage for other multiple AAV serotypes, such as AAV9, AAV2 and AAV1. This data provide support for using optimized SEC method to other AAV serotypes for the reliable quantitation of free DNA.

### Example 7

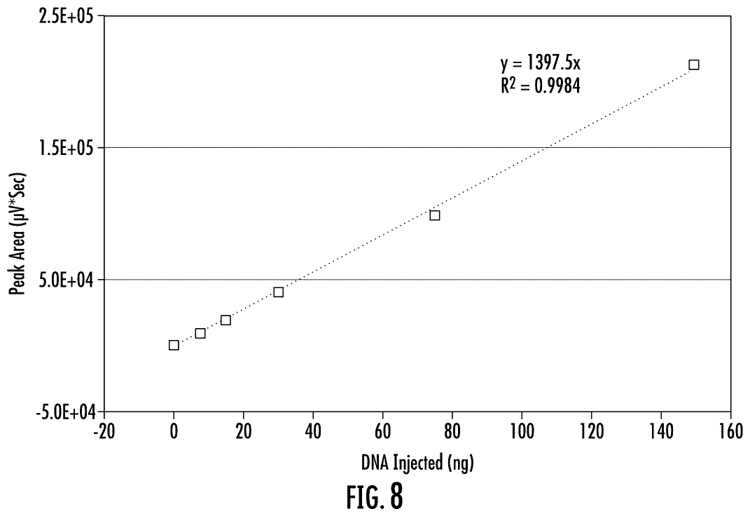

This Example shows the development of quantitation method for free DNA. To access whether the optimized SEC method can be applied to the quantify the free DNA. The correlation between the amount of injected DNA standard and the peak area measured with absorbance at 260 nm (FIG. 8) was first evaluated. To quantify the free DNA, a DNA standard is required to establish a calibration curve. By comparing the commercially available dsDNA and ssDNA standards, a 2 kb dsDNA standard was selected due to its high purity (100%). The results from this experiment demonstrated a strong linear relationship between the amount of DNA and the measured peak area, achieving a coefficient of determination (R2) of 0.9991 after linear regression analysis as shown in the equations disclosed in Example 8.

Using the calibration curve generated with the dsDNA standard, a formula based on Beer's Law was developed for calculating the amount of released free DNA. As shown in the equations disclosed in Example 8, a conversion factor using the ratio between the molar absorptivity of dsDNA and ssDNA was introduced into the formula to account for the fact that dsDNA was used to generate the calibration curve whereas in the study ssDNA was released from the AAV capsids. Applying this formula, the amount of released free DNA was calculated to be approximately 4 μg/mL from the AAV8 sample at 3E+13 vg/mL after 10 cycles of freeze/thaw treatment, which correlated to about 6% of the total genome DNA amount in the AAV sample. The presence of a small amount of contaminants in the dsDNA standard can contribute to the total absorbance at 260 nm, leading to a slightly smaller calculated free DNA values based on the calibration curve generated using the dsDNA standard. However, the values reported herein using this free DNA quantitation method provide valuable information for developing the risk assessment and mitigation strategies to achieve robust and stable AAV products.

### Example 8

This Example concerns an estimated concentration calculation of double stranded DNA based upon Beer's law, A=εbC. A is absorbance, ε is molar absorptivity (L/(mol*cm), b is light path length in cm, and C is concentration in mol/l. See Gallagher, Quantification of DNA and RNA with Absorption and Fluorescence Spectroscopy, Current Protocols in Cell Biology (2000).

The equations used are set forth below:

$$\begin{matrix}1. & Area ( dsDNA ) = {ε}_{2} bC 1 \\ 2. & C 1 = \frac{\frac{Area}{1397.5}}{{V}_{in j}} = \frac{Area}{{ε}_{1} b} \\ 3. & Area ( ssDNA ) = {ε}_{2} bC 2 \\ 4. & C 2 = \frac{Area}{{ε}_{2} b} = \frac{{ε}_{1}}{{ε}_{2}} ⋆ C 1\end{matrix} \begin{matrix}\to & C 2 = \frac{{ε}_{1}}{{ε}_{2}} ⋆ \frac{\frac{Area}{1397.5}}{{V}_{in j}}\end{matrix}$$

$$Note that :$$

$${ε}_{1} = 0.02 {( μg / ml )}^{- 1} {cm}^{- 1} ( dsNA )$$

$${ε}_{2} = 0.027 {( μg / ml )}^{- 1} {cm}^{- 1} ( ssDNA )$$

$${V}_{ing} is volume of injection$$

FIG. 8 depicts mass of 2 kb dsDNA injected vs. liquid chromatography (LC) area showing a good linear relationship between Peak Area (μV*Sec) and mass of DNA injected (ng). This conclusive linear formula permits the calculation of ssDNA estimated concentration.

### Example 9

This Example concerns an estimated concentration calculation of single stranded DNA based upon Beer's law. See Example 8. Recombinant AAV8 virions at a concentration of 3×10¹³ vg/ml were subjected to 10 cycles of Freeze/Thaw, which stresses the viral capsids, results in release of DNA. Injection volumes of 5 μl, 10 μl, 15 μl, 20 μl, 25 μl, 30 μl, 35 μl and 40 μl were tested. See Table 4.

TABLE 4

| | Average Free DNA | |
|---|---|---|
| Injection volume (μL) | Peak Area (μV*Sec) | Concentration (μg/mL) |
| 5 | 3.73 × 10⁴ | 3.95 |
| 10 | 7.45 × 10⁴ | 3.95 |
| 15 | 1.13 × 10⁵ | 3.99 |
| 20 | 1.49 × 10⁵ | 3.96 |
| 25 | 1.88 × 10⁵ | 3.98 |
| 30 | 2.24 × 10⁵ | 3.96 |
| 35 | 2.62 × 10⁵ | 3.97 |
| 40 | 2.99 × 10⁵ | 3.96 |

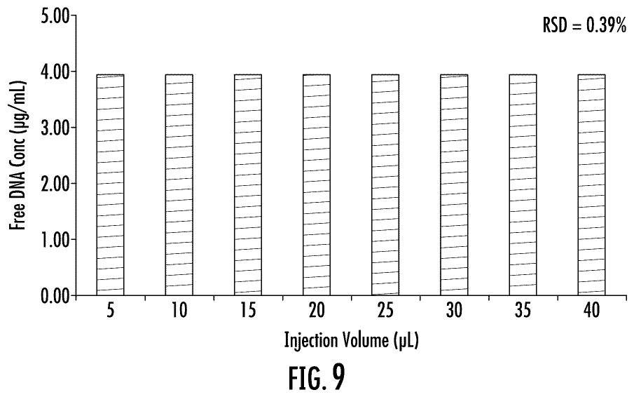

FIG. 9 is a bar graph depicting the calculation of free AAV8 DNA concentration (μg/ml) under different injection volumes (μl). Concentration was based on:

$$C = \frac{{ε}_{1}}{{ε}_{2}} ⋆ \frac{\frac{Area}{1397.5}}{{V}_{in j}}$$

### Example 10

This Example tested the capability of the method for AAV5. Replicate vials were used under each condition. Replicate running on LC (whole sequence replicate) were also carried out. Similar trending as for the AAV8 was observed. Results depicted in a bar graph in FIG. 10.

### Example 11

This Example provides an application of the SEC-UPLC methods to test the percentage of full capsids and formation of AAV dimers with SEC method

Since the system for the optimized SEC method was equipped with both a fluorescence detector and a PDA detector capable of performing triple-wavelength detection, experiments performed to explore whether the signals collected during the SEC analysis can be used to characterize additional quality attributes of AAVs in addition to free DNA. Three UV absorbance wavelengths at 230, 260 and 280 nm were selected due to the strong absorbance of peptide bonds at 230 nm, the high absorbance of nucleic acids at 260 nm, and the dominant absorbance of capsid proteins at 280 nm.

UV-Vis was used for assessing % Full for AAVs based on the understanding that there is a linear relationship between the absorbance ratio at 260 nm/230 nm and the percentage of full capsids (% Full) of AAVs. However, the drawback with a batch analysis method such as UV-Vis is that any impurity present in the sample can potentially interfere with the results. In this context, the optimized SEC method efficiently separates the AAVs from the free DNA and other impurities, so that the interference from impurities can be significantly minimized. Therefore, experiments were carried out to evaluate whether a linear correlation can be established between the absorbance ratio at 260 nm/230 nm for the main AAV particle peak and the % Full of AAVs using the SEC free DNA method.

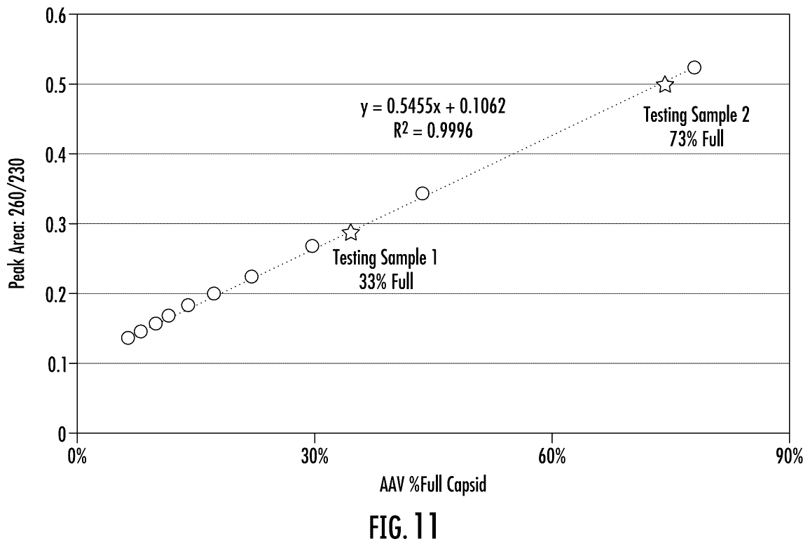

Two AAV5 samples, sample 1 with a low % Full of 6.3% and sample 2 with a higher % Full of 77.8%, were mixed at different ratios (Table 5) in order to generate a series of AAV5 samples with varying theoretical % Full. The % Full values in the two AAV5 reference standards were determined using mass photometry (MP), an orthogonal method to AUC for quantifying % Full capsids and the Empty to Full ratio of AAVs. The series of AAV5 samples with varying % Full prepared above were analyzed using the SEC method to create a calibration curve. The absorbance ratio at 260 nm/230 nm for the integrated area under the curves for the main AAV peak was determined and plotted against the theoretical % Full value calculated for each AAV5 sample (Table 5), which demonstrated a strong linear correlation with R2 of 0.9996 (FIG. 11). To confirm the SEC method for determination of % Full for AAV samples, two AAV5 samples with unknown % Full were analyzed using the SEC method. Based on the calibration curve generated using the two AAV5 reference standards as described herein, the % Full for these two AAV5 samples were determined to be 32.6% and 73.1%, respectively (FIG. 11). The same two AAV5 samples were subsequently analyzed using mass photometry and similar % Full values were obtained. These results provided strong evidence that the optimized SEC method has the potential to provide quantitative analysis of % Full for AAV samples. The % Full values reported using the SEC-free DNA method were demonstrated to be highly comparable to those reported using existing methods such as mass photometry which is commonly used for % Full characterization of AAVs.

Quantitation of % Full using the SEC free DNA method is provided in Table 5. Range of the theoretical % Full of the AAV samples used to create the calibration curve for quantitation of % Full.

TABLE 5

| AAV Sample 1:Sample 2 | % Full | 260 nm/230 nm |
|---|---|---|
| 10:0 | 6.30% | 0.138719 |
| 9:1 | 7.24% | 0.143702 |
| 8:2 | 8.38% | 0.149704 |
| 7:3 | 9.80% | 0.158468 |
| 6:4 | 11.60% | 0.168744 |
| 5:5 | 13.96% | 0.18448 |
| 4:6 | 17.21% | 0.201702 |
| 3:7 | 21.94% | 0.22692 |
| 2:8 | 29.49% | 0.270908 |
| 1:9 | 43.43% | 0.345208 |
| 0:10 | 77.8% | 0.52772 |

### Example 12

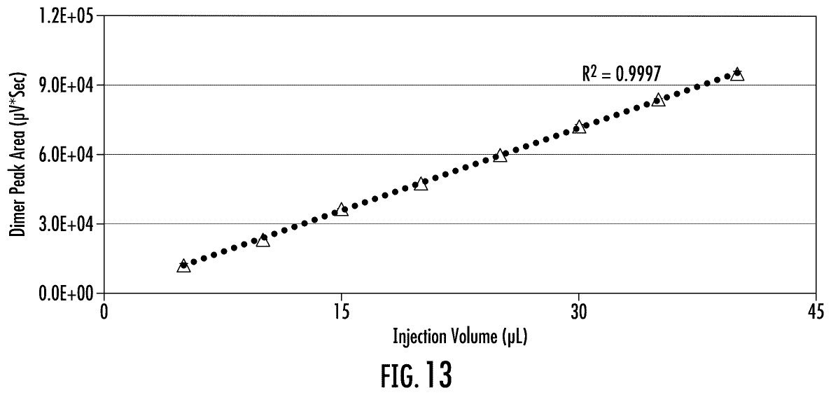

This Example evaluated SE-UPLC method performance for AAV dimer quantitation. One AAV5 sample containing identified dimer was analyzed using this SEC free DNA method. Experiments were performed in duplicate using serial injections of 5 μL to 40 μL. Results indicate that the method achieved good sensitivity, precision (% CV<6%) and linearity (R2 is >0.999). FIG. 13 depicts the linearity of dimer peak area.

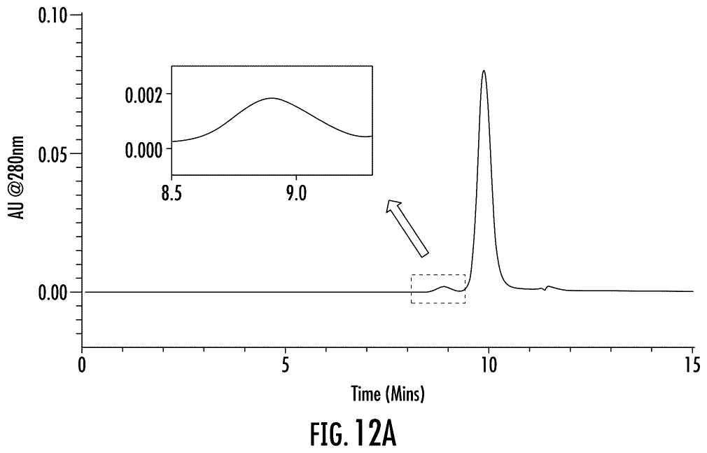

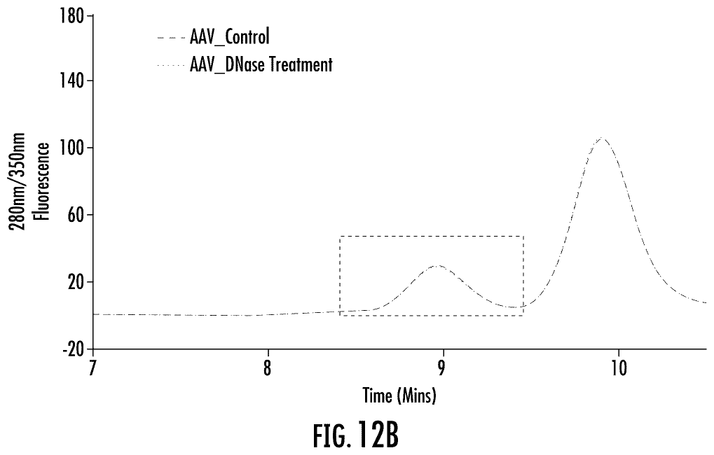

For biologics analysis, SEC is can be used for the characterization of smaller soluble aggregates, or high molecular weight (HMW) species, such as dimers and oligomers. Thus, experiments were performed to assess whether the SEC free DNA method can be applied to the characterization of HMW species in AAV samples. One AAV5 sample containing identified dimer was analyzed using this SEC free DNA method. An additional peak eluting at about 9 minutes was observed, which was well separated from the free DNA peak eluting at about 7 minutes and the main AAV particle peak eluting at about 9.5 minutes (FIG. 12A). The additional peak found to exhibited positive fluorescence signal and remained unchanged following DNase treatment (FIG. 12B), which further confirmed that the additional peak was due to formation of AAV5 dimers instead of DNA impurities. Then, the method performance for quantitation of dimers in AAV5 samples was evaluated. A series of injections of the AAV5 sample with increasing injection volumes from 5 μL to 40 μL were performed to generate a calibration curve with the absorbance at 230 nm for the integrated area under the curves for the AAV dimer peak plotted again the injection volumes (FIG. 13). Wavelength 230 nm was selected over 280 nm for the quantitation of AAV dimers because the absorbance intensity of the AAV dimers at 230 nm was significantly higher than that at 280 nm resulting in much larger signal-to-noise ratio. The results demonstrated that the SEC free DNA method exhibits good sensitivity, precision, and linearity for the quantitation of dimers in the AAV5 samples. The methods, as disclosed herein, also can be applied to other AAV serotypes for the quantitation of dimers or oligomers if present.

What is claimed is:

1. A method of analysing free DNA in a preparation comprising recombinant AAV (rAAV) viral particles, wherein the method comprises the steps of:
(a) loading the preparation comprising rAAV viral particles in a size exclusion chromatography column having a pore size greater than 500 angstroms, wherein prior to loading the preparation is mixed with a running buffer;
(b) running the preparation on the size exclusion chromatography column at a flow rate of less than 0.5 ml/minute, wherein the pressure in the size exclusion chromatography column does not accumulate and result in column clogging; and
(c) detecting the elution of free rAAV DNA and rAAV viral particles from the size exclusion chromatography column.

2. The method according to claim 1, wherein the size exclusion chromatography column media has a nominal pore size of at least about 700 angstroms.

3. The method according to claim 1, wherein the size exclusion chromatography column media has a pore size of at least about 900 angstroms.

4. The method according to claim 1, wherein the size exclusion chromatography column has a pore size of about 900-1100 angstroms.

5. The method according to claim 1, wherein the size exclusion chromatography column has a pore size of at least about 1000 angstroms.

6. The method according to claims 1-5, wherein the flow rate of step (b) is 0.45 ml/minute.

7. The method according to claims 1-5, wherein the flow rate of step (b) is 0.40 ml/minute.

8. The method according claims 1-5, wherein the flow rate of step (b) is 0.35 ml/minute.

9. The method according to claims 1-5, wherein the flow rate of step (b) is 0.30 ml/minute.

10. The method according to claims 1-9, wherein running buffer comprises a surfactant.

11. The method according to claims 1-10, wherein the running buffer has a total salt content of over 140 mM.

12. The method according to claims 1-10, wherein the running buffer has a total salt content of over 150 mM.

13. The method according to claims 1-10, wherein the running buffer has a total salt content of over 200 mM.

14. The method according to claims 1-10, wherein the running buffer has a total salt content of over 250 mM.

15. The method according to claims 1-10, wherein the running buffer has a total salt content of over 300 mM.

16. The method according to claims 11-15, wherein the running buffer comprises two types of salts.

17. The method according to claim 16, wherein the salts are sodium chloride and sodium phosphate.

18. The method according to claims 1-10, wherein the running buffer comprises a poloxamer.

19. The method according to claim 18, wherein the poloxamer is poloxamer 188.

20. A method of determining stability of rAAV comprising measuring capsid-free DNA using SE-UPLC at 260 nm UV and analysing the free-DNA according to claim 1.

21. The method according to claim 20, wherein the preparation of rAAV viral particles comprising a buffer, a cryoprotectant, a salt, a non-ionic surfactant at a pH of about 6.0 to about 8.0.

22. The method according to claim 21, wherein the cryoprotectant is sucrose at a concentration of about 0.5 to about 5.0% (w/v).

23. The method according to claim 21, wherein the concentration of sucrose is about 1.0 to about 3.5% (w/v), about 1.0 to about 3.0% (w/v), about 1.0 to about 2.5% (w/v), about 1.0 to about 2.0% (w/v), about 1.0 to about 1.5% (w/v), about 1.0% (w/v), about 1.5% (w/v), about 2.0% (w/v), about 2.5% (w/v), about 3.0% (w/v).
